(19) United States
(12) Patent Application Publication
(10) Pub. No.: US 2009/0149536 A1
(43) Pub. Date: Jun. 11, 2009

(54) 4-OXO-(ISO)TRETINOIN FOR THE TOPICAL TREATMENT OF SEVERE DERMATOLOGICAL DISORDERS

(76) Inventors: Pierre Barbier, Rixheim (FR); Jutta Heim, Ramlinsburg (CH); Anne Hoffmann Schmitt, Basel (CH)

Correspondence Address: GIBBONS P.C., ONE GATEWAY CENTER, NEWARK, NJ, 07102, US

(21) Appl. No.: 12/132,998

(22) Filed: Jun. 4, 2008

Related U.S. Application Data
(63) Continuation-in-part of application No. PCT/CH2006/000689, filed on Dec. 8, 2006.

(30) Foreign Application Priority Data
Dec. 9, 2005 (EP) 05026949.7

(51) Int. Cl. A61K 31/19; A61P 17/00; A61P 17/10

(52) U.S. Cl. 514/557

(57) ABSTRACT

A method for the treatment of dermatological disorders wherein such treatment includes at least one of inhibiting lipogenesis in the skin and reducing the size of sebaceous glands, said treatment comprising the topical administration of a compound selected from 4-oxo-tretinoin, 4-oxo-isotretinoin and mixtures thereof to a patient in need thereof. The method of the invention is particularly advantageous for the topical treatment of severe acne and seborrhoea.

### RELATED APPLICATIONS

This application is a continuation-in-part of International Patent Application Serial No. PCT/CH2006/000689 filed 8 Dec. 2006, which claims priority from European Patent Application No. 05026949.7, filed on 9 Dec. 2005.

### BACKGROUND OF THE INVENTION

The present invention relates to the use of the retinoids 4-oxo-all-trans-retinoic acid (4-oxo-tretinoin) and 4-oxo-13-cis-retinoic acid (4-oxo-isotretinoin) for the treatment of severe dermatological disorders, in particular for the treatment of severe acne and seborrhoea.

Acne is known to be caused by interactions between hormones, skin oils, and bacteria that result in inflammation of hair follicles. Acne occurs mostly on the face, upper chest, shoulders, and back and is characterized by pimples and sometimes cysts and abscesses which may be pus-filled.

Sebaceous glands, which secrete an oily substance (sebum), lie in the dermis, the middle layer of skin. These glands are attached to the hair follicles. The sebum, along with dead skin cells, passes up from the sebaceous gland and hair follicle and out to the surface of the skin through the pores.

Acne results when a conglomerate of dried sebum, dead skin cells, and bacteria clog the hair follicles, blocking the sebum from leaving through the pores. If the blockage is incomplete, a blackhead (open comedone) develops; if the blockage is complete, a whitehead (closed comedone) develops. The blocked sebum-filled hair follicle promotes overgrowth of the bacteria Propionibacterium acnes, which are normally present in the hair follicle. These bacteria break down the sebum into substances that irritate the skin. The resulting inflammation and infection produce the skin eruptions that are commonly known as acne pimples. If the infection worsens, an abscess may form, which may rupture into the skin, creating even more inflammation.

Acne naturally varies in severity from mild to very severe. People with mild (superficial) acne develop only a few noninflamed blackheads or a moderate number of small, mildly irritated pimples, mostly on the face. Blackheads appear as tiny, dark dots at the center of a small swelling of normal-colored skin. Pimples are mildly uncomfortable and have a white center surrounded by a small area of reddened skin. People with severe (deep, or cystic) acne, on the other side, have numerous large, red, painful pus-filled lumps (nodules) that sometimes even join together under the skin into giant, oozing abscesses.

Mild acne usually does not leave scars. The nodules and abscesses of severe acne often rupture and, after healing, typically leave scars. Scars may be tiny, deep holes (ice pick scars); wider pits of varying depth; or large, irregular indentations. Acne scars last a lifetime and are frequently cosmetically significant.

A treatment option for acne are retinoids, which have been widely described in the past for treatment of a number of dermatological disorders, including acne and seborrhoea.

Topical all-trans retinoic acid (tretinoin) like creams or gels have been commercialized for the treatment of mild forms of acne but have proved to be not suitable for more severe forms of acne and seborrhoea.

For an appropriate treatment of such severe forms of seborrhoea and particularly acne, oral administration of retinoids turned out to be necessary. Orally administered 13-cis retinoic acid (isotretinoin) revolutionized the treatment of severe forms of acne when it was introduced in 1982, and continues the present to be the single therapy capable of curing severe acne. Oral isotretinoin is so effective against acne because it is the only treatment affecting all major aetiological factors, including, in particular, a substantial reduction of sebum production by inhibition of the lipogenesis, as well as a reduction of the size of sebaceous glands of the patient. Thus, oral isotretinoin was established in the last decade as the gold standard of acne therapy, capable of long-term remission in 80% of patients with severe acne.

Isotretinoin, however, like many other retinoids is known to possibly cause several serious side-effects, in particular when administered systemically, like birth defects; mental health problems including suicide danger; uncontrolled increases of brain pressure which can e.g lead to permanent loss of sight; damage of liver, pancreas, bowel and oesophagus; possible bone and muscle problems; development of high levels of cholesterol and other fats in the blood; and others. That is why systemic isotretinoin is usually applied only for treatment of severe forms of acne that cannot be cleared up by other acne treatments, for example, systemic antibiotics, e.g for the treatment of severe nodular acne, where the possible benefits of isotretinoin are considered to outweigh its risks.

Retinoid formulations for topical administration would certainly represent an improvement with regard to the risk of possible side effects because of the substantially shorter way of the drug from the point of administration to the point of action associated with topical administration that results in a lower systemic exposure. Topical retinoids, however, have only a comedolytic effect. They are substantially effective in normalizing the desquamation of the follicular epithelial layer, thus improving the outflow of already existing comedones and preventing the formation of new microcomedones and restoring the follicular micro-environment. Neither topical tretinoin nor topical isotretinoin have been found to have an influence on the dermal lipogenese and sebum production (Ch Zouboulis, “Modern acne treatment”, Akt Dermatol. 2003; 29: 49-57).

Compared with mild acne the more severe forms of acne, however, are known to be strongly characterized by an increased lipid biosynthesis in the dermis and epidermis of the affected skin (S. Shuster; M F Cooper; D. McGibbon; P D Wilson; “Epidermal lipid biosynthesis in acne”; Br J Dermatol, 1980; 103: 127-130).

In accordance with the aforementioned facts, it has furthermore been known in the art that, contrary to oral isotretinoin, topical isotretinoin is not useful for treatment of severe acne and seborrhoea. This is because it is recognized that the effectiveness of isotretinoin after topical administration in reducing sebaceous gland size and in suppressing sebum production is reduced by up to 90% as compared to the effect obtained after oral administration of isotretinoin. The low topical efficacy of isotretinoin could indicate that metabolites of isotretinoin which are effective for creating the aforementioned pharmacological effects after oral administration are not formed in the physiological environment of the skin.

As regards the metabolites of isotretinoin, it is furthermore known that 4-oxo-isotretinoin and its equilibrium compound under physiological conditions, 4-oxo-tretinoin, are the major metabolites of isotretinoin, when said compound is administered systemically.

Furthermore, 4-oxo-isotretinoin and 4-oxo-tretinoin have already been suggested for use as pharmaceutical agents. U.S. Pat. No. 4,169,103 describes the use of 4-oxo-tretinoin and 4-hydroxy-tretinoin for reduction of the growth of tumors in mammals. Without actual relation to said main subject matter of this reference, it furthermore mentions that 4-oxo-tretinoin and 4-hydroxy-tretinoin should also be useful for the systemic and topical therapy of acne, psoriasis and other dermatological disorders characterized by increased or pathologically altered cornification as well as of inflammatory and allergic dermatological conditions. Except for this unrelated disclosure, however, the reference is completely silent with regard to the treatment of dermatological conditions and it does, in particular, not mention anything about a possible effect of 4-oxo-tretinoin or 4-hydroxy-tretinoin on sebaceous gland size and pathological dermal lipogenesis associated with severe forms of acne.

Furthermore, GB-A-2 156 676 discloses that 4-oxo-isotretinoin and 4-hydroxy-isotretinoin can be used as active component of pharmaceutical and cosmetic formulations, suitable for the treatment of acne and seborrhoea. The preferred administration route is oral, although a topical administration in form of a solution, lotion or a hair shampoo is also mentioned. There is no indication whatsoever in said reference as to the degree of severity of the acne and/or seborrhoea which is actually envisaged for treatment with said compounds, and the reference does again not mention anything about a possible effect of 4-oxo-tretinoin or 4-hydroxy-tretinoin on sebaceous gland size and pathological dermal lipogenesis associated with severe forms of acne.

Very recently, it has furthermore been shown that 4-oxo-isotretinoin reduces the sebum excretion rate when applied orally (Canadian Dermatology Association Meeting 2003). 4-Oxo-isotretinoin, however, has been found to be only half as active pharmacologically as its parent drug isotretinoin (Ulf-W Wiegand (Roaccutane®—Important New Data for Efficacy and Safety Business Briefing: European Pharmacotherapy 2003).

### SUMMARY OF THE INVENTION

It has now been found that topically administered 4-oxo-tretinoin and 4-oxo-isotretinoin inhibit the lipogenesis in the skin and/or are effective in reducing the sebaceous gland size, both to an extent which is at least comparable to orally administered isotretinoin, the present gold standard for treatment of severe acne.

It has furthermore been found that 4-oxo-tretinoin and 4-oxo-isotretinoin when topically adiministered to the skin only generate a surprisingly low irritation of the skin, a problem with almost all retinoids (including isotretinoin itself) when topically applied.

### DETAILED DESCRIPTION OF THE INVENTION

Therefore, the present invention relates to the use of a compound selected from the group 4-oxo-isotretinoin, 4-oxo-tretinoin and mixtures thereof for the manufacture of a medicament for topically administration, inhibiting the lipogenesis in the skin and/or reducing the sebaceous gland size of a patient in need thereof.

In another aspect, the present invention concerns the use of a compound selected from the group consisting of 4-oxo-isotretinoin, 4-oxo-tretinoin and mixtures thereof for the manufacture of a medicament for the topical treatment of severe acne and seborrhoea.

Acne, in particular acne vulgaris, is generally divided into three forms, namely mild, moderate or severe acne. Acne is a frequently chronical disease of the sebaceous follicles which is multifactoral in ethiology and occurs particularly on the face, but also on non-facial skin, e.g. back, shoulders or chest.

Whereas mild and moderate acne is mainly characterized by the sole presence of so called white heads, closed comedos, which occur when sebum accumulates beneath a thin layer of superficial skin, and blackheads, or open comedos, resulting from follicle being spread open by a core of the keratinocytes that appear dark due to oxidation, but substantially no inflammation, severe forms of acne are mainly characterized by inflammation and the presence of lesions, in particular papules, pustules, nodules, or cysts.

A papule is a raised lesion 1.0 cm or less in diameter, while a pustule is a similar lesion containing pus. Nodules are lesions that are 1.0 cm or larger, often extending deeper or higher, and often firm to the touch. Cysts are nodules filled with fluid, often pus.

Severe acne causes strongly visible scaring and will cause deep, inflammatory nodules. Another name for this condition is cystic acne, which is mainly characterized by numerous comedones, papules, and pustules, spreading to the back, chest, and shoulders, numerous large cysts or nodules on the face, neck, and upper trunk, and severe scarring. Among the sub-classes of severe acne are:

Acne conglobata is a chronic form of severe acne showing deep abscesses, strong inflammation, severe damage to the skin, extensive scarring; inflammatory nodules forming around multiple comedones and growing until they break down and discharge pus; deep ulcers forming under the nodules, producing keloid-type scars, and crusts forming over deeply ulcerated nodules. Abscesses can form deep, irregular scars. Acne conglobata may be preceded by acne cysts, papules or pustules that do not heal, but instead rapidly deteriorate, and occasionally may flare up in acne that had been dormant for many years.

Nodulocystic acne is severe acne with cysts. They may occur in isolation or be widespread over the face, neck, scalp, back, chest and shoulders. They can be very painful. Acne cysts are nodules of inflammation. A cyst may be filled with thick, yellow pus-like fluid and is inflamed and infected. If such a cyst is drained, it must be done under sterile conditions.

The guidance for Industry Acne Vulgaris: Developing Drugs for Treatment of the U.S. Department of Health and Human Services Food and Drug Administration (CDER) September 2005 and the Report of the Consensus Conference on Acne Classification, Washington, DC Mar. 24 and 25 1990. J Am Acad Dermatol 1991; 24: 495-500 contain further details with regard to the distinction of severe acne from mild to moderate forms.

Furthermore, several grading systems exist for classification of the acne into mild, and moderate forms as compared to severe acne, one relatively new one being the Leeds revised acne grading system, which is described in the Journal of Dermatological Treatment (1998) 9, 215-220 by S C O'Brian; J B Lewis and W J Cunliffe. The Leeds grading system distinguishes facial acne into 12 grades, with grade 12 being the most severe; and acne on back and chest into 8 grades, with grade 8 being the most severe.

For the purposes of this application, the term “severe acne” shall be understood to mean preferably those forms of acne which have so far conventionally been treated with systemic, in particular with oral, isotretinoin, for example, Accutane or Roaccutane. These forms of acne include cystic nodular acne, acne conglobata, acne fulminans, acne inversa and those forms of acne vulgaris, in which any of the following characteristics is present: persistent or recurrent inflammatory nodules, extensive papulopustular disease, ongoing scaring, persistent purulent and/or serosanguineous drainage from lesions, or sinus tracks.

In terms of the Leeds revised acne grading system mentioned above, severe acne is understood to cover those forms of facial acne having a grade of 7 to 12 according to said grading system as well as those forms of acne on the back and chest having a grade of 5 to 8.

Seborrhoea is an excessively oily skin, due to overactive sebaceous glands and is usually associated with acne. For the purposes of this application, the term “severe seborrhoea” shall be, in particular, directed to seborrhoea associated with severe forms of acne as defined hereinabove.

In a preferred embodiment of the present invention, 4-oxo-isotretinoin and 4-oxo-tretinoin are used for the manufacture of a medicament for the treatment of severe acne, in particular facial acne of a grade of 7, more specifically of 9 or more according to the Leeds revised acne grading system, or acne at the back or chest of a grade of 5, more specifically 6 or more, according to said system.

Further preferred embodiments are the use of 4-oxo-isotretinoin and 4-oxo-tretinoin for the manufacture of a medicament for the treatment of acne conglobata or the use for the manufacture of a medicament for the treatment of nodulocystic acne. A specific embodiment of the present invention is furthermore the use of 4-oxo-tretinoin for the manufacture of a medicament for the topical treatment of severe acne and seborrhoea.

A further embodiment is the use of 4-oxo-isotretinoin or a mixture of 4-oxo-isotretinoin and 4-oxo-tretinoin, preferably 4-oxo-isotretinoin, for the manufacture of a medicament for the topical treatment of severe acne and seborrhoea.

Preferably, 4-oxo-isotretinoin and 4-oxo-tretinoin are applied at dosages which do not significantly increase the endogenous plasma level of these compounds which is about 4.85±3.89 ng/mL (n=50) for 4-oxo-isotretinoin and about 9.88±7.57 (n=48) for 4-oxo-tretinoin in man, in order to keep adverse effects of said retinoids as low as possible. In a specific embodiment of the present invention, the dosages are selected such that they do not elicit teratogenicity in animals even when strongly exceeded. Suitable dosages are described herein below and can be determined by a doctor based on usual parameters like e.g. the weight and age of the patient, and the current status of the disease.

4-Oxo-isotretinoin and/or 4-oxo-tretinoin may be used according to the invention in form of a topical composition that may e.g. be in the form of a solution, cream, ointment or a gel. These compositions may additionally comprise a cosmetically acceptable vehicle to act as diluent, dispersant or carrier for the active components in the composition, so as to facilitate their distribution when the composition is applied to the skin. The vehicles can also comprise further excipients usual for topical application forms like e.g. liquid or solid emollients, solvents, humectants, thickeners, emulsifiers, fillers and the like. The concentration of the active components in the composition of the invention ranges preferably from about 0.001% to about 1%, more preferably from about 0.025% to about 0.2%, most preferably about 0.05% to about 0.15%. A typical example is a topical composition comprising about 0.1% of 4-oxo-isotretinoin and a pharmaceutically acceptable carrier and optionally one or more excipients.

It has been found that such concentrations of 4-oxo-isotretinoin and/or 4-oxo-tretinoin in the a topically applicable medicament do not cause a major skin irritation when administered in a suitable dosage regimen, e.g. once to several times, e.g. one to four times, per day to the skin of an adult of 50 to 120 kg weight and for a suitable time period e.g. four weeks to several month. A preferred dosage will consist of administration once to twice a day of approx. 0.5 g to 1 g of cream containing an effective quantity of up to 0.5% of 4-oxo-tretinoin or up to 1% of 4-oxo-isotretinoin per areas (10×10 cm).

The following Examples serve to illustrate the invention without limiting the scope thereof in any particular.

### EXAMPLE 1

### Typical Formulations

Oil-in-Water Cream

| Component | Amount in Grams |
|---|---|
| Active substance | 0.1 g |
| Emulsifying Cetostearylalcohol | 9.0 g |
| Liquid paraffin | 10.5 g |
| Vaseline | 10.5 g |
| Water | 69.9 g |

Ointment

| Component | Amount in Grams |
|---|---|
| Active substance | 0.1 g |
| Wool (lanolin) alcohols | 3.0 g |
| Cetostearylalcohol | 0.25 g |
| Vaseline | 46.75 g |
| Water | 49.9 g |

Aqueous Gel

| Component | Amount in Grams |
|---|---|
| Active substance | 0.1 g |
| Butylated hydroxytoluene | 0.02 g |
| Hydroxypropylcellulose | 2.0 g |
| Ethanol 99.5% | 97.07 g |

These formulations are prepared in accordance with conventional procedures known to those skilled in the art. The in vitro genotoxic potential of 4-oxo-tretinoin and 4-oxo-isotretinoin is negative in the micro-Ames test and in the MNT test.

### EXAMPLE 2

### Efficacy Determination

The cliniclal efficacy of the compounds 4-oxo-tretinoin and 4-oxo-isotretinoin used according to present invention can be shown by a person skilled in the art with usual clinical trials, performed, for example, according to following protocol synopsis:

| Indication | Severe cystic acne |
|---|---|
| Objectives | To estimate the plasma concentration of 4-oxo- |
| | tretinoin and 4-oxo-isotretinoin following topical |
| | treatment with 4-oxo-tretinoin or 4-oxo- |
| | isotretinoin |
| Study design | Open, comparative, sequential pilot trial, with a |
| | total duration of 4 to 8 weeks |
| Planned total sample | 10 volunteers or 10 patients |
| size | |
| Subject selection | Volunteers with hyperseborrhoic skin or patients |
| criteria | with hyperseborrhoic skin and clinical signs of |
| | acne: male, aged 18-65; female, if they are |
| | physiologically or physically sterile |
| Formulations | 0.05% up to 1% Cream: 4-oxo-tretinoin or 4-oxo- |
| | isotretinoin, 30 g applied on the back |
| Concurrent control | Topical administration: 4-oxo-tretinoin or 4-oxo- |
| | isotretinoin (placebo), 30 g, applied on the back. |
| Test drug dosage | Approx. 2 g cream (±1.5 cm³) b.i.d. for 4 to 8 |
| | weeks |
| Route of | Topical |
| administration | |
| Main parameters of: | |
| Efficacy | Reduction of sebum secretion rate in healthy |
| | volunteers or clinical rating of acne in patients |
| Safety | Adverse events, laboratory tests |
| Pharmacokinetics | Plasma concentration of 4-oxo-tretinoin and 4- |
| | oxo-isotretinoin |

Procedure:
Volunteers or patients will apply twice-daily approx. 2 g of cream (±1.5 cm³) containing up to 0.5% of 4-oxo-tretinoin or up to 1% of 4-oxo-isotretinoin, on the designated areas of the back (20 × 35 cm) for duration of 4 weeks. Clinical efficacy will be evaluated by the reduction of sebum excretion rate or the overall clinical rating of acne. Blood samples will be taken at trough during week 4 or 8 to estimate steady state plasma concentration. During the entire study the subjects should avoid exposure to sun.
Statistical analysis:
Only descriptive statistics will be employed.

### EXAMPLE 3

### Efficacy Determination in Severe Acne

Fifteen male patients aged between 18 and 65 with severe acne on back or chest rated a grade of 5 to 8 according to the Leeds revised acne grading system (Journal of Dermatological Treatment (1989) 9, 215-220) are enrolled in a pilot study.

Each patient receives 2 g of a cream containing 0.5% of 4-Oxotretinoin or up to 1% of 4-Oxo-Isotretinoin topically at a suitable dosage regimen, e.g. one to four times per day, on a designated area of the back (20×35 cm) for a total time of 4 weeks at minimum to several months. After completion of the treatment, the grade of the acne in the designated treatment area is once more determined and compared with the surrounding area of the skin.

The topical treatment with 4-Oxotretinoin and 4-Oxoisotretinoin reduces the clinical rating of the acne in the treated area significantly when compared with the untreated skin outside of the treated area.

### EXAMPLE 4

### Animal Trials

Fuzzy rats can be distinguished at birth from phenotypically normal siblings by their curled vibrissae and from day 10-15 on their initially sparse pelage with short, broken hairs resulting in a wavy coat which then becomes rapidly devoid of hair. At about 2 months of age, the phenotypic appearance of mutants ranges from a sparse fuzzy coat to almost complete hairlessness. On histological examination, the skin is poorly developed with various sized cystic hair follicles containing concentric lamellar accumulations of keratinacious material, which are often associated with enlarged sebaceous glands. Two variants displaying the same type of hypotrichosis have been described (Ferguson et al., Lab. Anim. Sci., 29: 459-465, 1979).

Accordingly, this model of rat is well appropriated for studying the influence of a test compound on lipogenesis in the skin and sebaceous gland size and thus the sebosuppressive effect of said test compound. By the way of example, Puhvel et al., Arc. Dermatol. Res., 277: 395-399, 1985 have studied the efficacy of 13-cis-retinoic acid after topical administration to the skin in the Fuzzy rat model and have found that, at non-toxic dosage, topical 13-cis-retinoic acid exhibits substantially no detectable sebosuppressive activity.

### Material and Methods

### Test Substances, Reference Substances and Vehicle:

The test substances are light sensitive, so they were manipulated in a room lit by low intensity light (<100 Lux).

4-Oxo-tretinoin was received aliquoted in 25 vials containing solution of 0.2% 4-oxo-tretinoin in Aceton/ethanol 1/1 and 25 vials containing solution of 0.1% 4-oxo-tretinoin in Aceton/ethanol 1/1. Each vial was stored at −20° C.

The test substance 4-oxo-isotretinoin was received aliquoted in 25 vials containing solution of 0.5% 4-oxo-isotretinoin in Aceton/ethanol 1/1 and 25 vials containing solution of 0.1% 4-oxo-isotretinoin in Aceton/ethanol 1/1. Each vial was stored at −20° C.

The positive reference, 13-cis-retinoic acid, was received aliquoted in 24 amber glass injection vials, filled each with 11 ml of 13-cis-RA at 2 mg/ml suspended in olive oil with 0.01% alpha-Tocopherol. Each vial was stored at −20° C.

The vehicle Aceton/ethanol 1/1 was received in 2 clear glass 25 ml lab bottles and stored at room temperature.

### Radioactive Elements

The [4-¹⁴C]-Cholesterol (Ref CFA128-50UCI, batch No. 151, 50 μCi, 1.85 MBq, 3.7 MBq/ml, 100 μCi/ml, Amersham Biosciences, France) radiolabeled ligand was purchased and dissolved in toluene solution. The specific activity was 58 mCi/mmol. The compound was received, stored and manipulated according to supplier's instructions.

The D-[U-¹⁴C]-Dextrose (Ref. CFB96-1MCI, batch No. 223, 1 mCi, 37 MBq, 7.4 MBq/ml, 200 μCi/ml, Amersham Biosciences, France) radiolabeled ligand was purchased and dissolved in aqueous solution containing 3% ethanol. The specific activity was 281 mCi/mmol. The compound was received, stored and manipulated according to supplier's instructions.

### Doses and Administration Route

4-oxo-isotretinoin was used in aliquots ready-to-use at two concentrations: 0.1% and 0.2% (one aliquot/dose/day of treatment). It was administered by topical application of 25 μl/2 cm² using 25 μl pipettes Microman Gilson.

4-oxo-isotretinoin was used in aliquots ready-to-use at two concentrations: 0.1% and 0.5% (one aliquot/dose/day of treatment). It was administered by topical application of 25 μl/2 cm² using pipettes Microman Gilson.

The positive reference, 13-cis-retinoic acid, was used in soybean wrap oil at 2 mg/ml and administered by oral route (PO) at 5 ml/kg, the administration dose being 10 mg/kg. The vehicle [ethanol:acetone (1:1; v:v)] was administered by topical application of 25 μl/2 cm² using pipettes Microman Gilson.

Eighteen healthy female Fuzzy rats, 11 weeks old, were obtained from Harlan (Gannat, France). The animals were maintained for 7 days in a conventional and officially authorized animal care unit before the start of the study. Animal experiments were performed according to ethical guidelines of animal experimentations (Principe d'éthique de l'expérimentation animale, Directive n°86/609 CEE du 24 Nov. 1986, Décrêt n°87/848 du 19 Oct. 1987, Arrêté d'Application du 19 Avril 1988).

The animals were maintained in rooms under controlled conditions of temperature (21±1° C.), humidity (60±5%), photoperiod (12 h light/12 h dark) and air exchange. Animals were maintained in conventional conditions; the room temperature and humidity were continuously monitored. The air handling system was programmed for 14 air changes an hour, with no recirculation. Fresh outside air passes through a series of filters, before being diffused evenly into each room. All personnel working under conventional conditions followed specific guidelines regarding hygiene and clothing when they entered in the animal husbandry area. From begin (Do) to end of the study, the room was lit by low intensity light (<100 Lux) because of the light sensivity of test substances.

Animals were housed in polycarbonate cages (UAR, Epinay sur Orge, France) that were equipped to provide food and water. The standard size cages used were 800 cm² with a maximum of 3 rats per cage according to internal standard operating procedures. Bedding for animals was sterile wood shavings DIETEX (Saint-Gratien, France), replaced twice a week.

Food was provided to the animals ad libitum, being placed in the metal lid on top of the cage. Water was also provided ad libitum from water bottles equipped with rubber stoppers and sipper tubes. Water bottles were cleaned, sterilized and replaced once a week.

Animal Treatment with 13-Cis-RA, 4-Oxo-Tretinoin and 4-Oxo-Isotretinoin

At Do, eighteen (18) Fuzzy female rats were randomized in 4 groups (5 rats/group for test substances and positive control, and 3 rats/group for vehicle group) according to body weight criteria:
one vehicle group treated by topical application of vehicle in a predefined patch of 2 cm² (one patch per rat), the same during all the treatment according to the treatment schedule (once a day, 5 days per week for 4 weeks, [(Q1DX5)×4 weeks]), (Group No. 1, mean body weight: 212.3±12.1 g)
one group treated orally (PO) with 13-cis-RA at 10 mg/kg according to the treatment schedule [(Q1DX5)×4 weeks]. (Group No. 2, mean body weight: 214.6±10.5 g)
one group treated with 2 concentrations of 4-oxo-tretinoin (0.1 and 0.2%) per rat. Each rat was treated by topical application of test substance on 2 predefined patches of 2 cm² according to the treatment schedule [(Q1DX5)×4 weeks]. (Group No. 3, mean body weight: 212.2±8.0)
one group treated with 2 concentrations of 4-oxo-isotretinoin (0.1 and 0.5%) per rat. Each rat was treated by topical application of test substance on 2 predefined patches of 2 cm² according to the treatment schedule [(Q1DX5)×4 weeks]. (Group No. 4, mean body weight: 213.8±11.5 g)

The experimental design is summarized in the Table below.

| | animals | | | | | |
|---|---|---|---|---|---|---|
| Test | per | | Administration | Administration | Treatment | Treatment |
| group | group | Treatment | route | volume | dose | schedule |
| 1 | 3 | Vehicle | cutaneous | 25 μl/2 cm² | 0.0% | [Q1DX5] |
| 2 | 5 | 13-cis-RA | PO | 5 ml/kg | 10 mg/kg | x4 weeks |
| 3 | 5 | 4-oxo- | cutaneous | 25 μl/2 cm² | 0.1%-0.2% | |
| | | tretinoin | | | | |
| 4 | 5 | 4-oxo- | cutaneous | 25 μl/2 cm² | 0.1%-0.5% | |
| | | isotretinoin | | | | |

Rats were observed for 2 h post-treatment. The viability, behavior and body weight of rats were recorded twice a week until the end of the experiment since no serious weight loss was observed. Weight loss was assessed against the starting weight of each rat.

During the course of the experiment, no animal was killed since nothing of the following occured:
Signs of suffering (cachexia, weakening, difficulty to move or to eat),
Compound toxicity (hunching, convulsions),
25% body weight loss on any day.

At the end of the 4-week treatment period, pentobarbital (Ref. P3761, batch No. 59H0612, Sigma; 70 mg/kg by intraperitoneal route (IP)) was used to anaesthetize the animals before sacrifice by inhalation of CO₂.

### Study of Lipogenesis

### Collecting of Biopsies

At the end of the treatment, animals were anaesthetized with Pentobarbital 70 mg/kg by IP route. For each rat, two biopsies of 3-mm of dorsal skin per treated region with test substances were collected. Each biopsy was weighed. Rats were sacrificed by inhalation of CO₂.

Half of biopsies was immediately transferred in (KRB) Krebs-Ringer buffer (NaCl 108 mM, Ref. H5271, batch No. 104176, Promega; KCl 4.74 mM, Ref. P3911, batch No. 112K3720, Sigma; MgSO₄ 1.19 mM, Ref M5921, batch No. 034K0066, Sigma; CaCl₂ 2.54 mM, Ref. C7902, batch No. 044K0160, Sigma; KH₂PO₄ 1.19 mM, Ref P5379, batch No. 024K0050, Sigma; NaHCO₃ 18 mM, Ref. S6297, batch No. 082K0125, Sigma; Bovine Serum Albumine 0.2%, Ref. A9418, batch No. 084K0578, Sigma; D-Glucose 8.3 mM, Ref. G7528, batch No. 033K0121, Sigma) for further analysis of lipogenesis.

The other biopsies were transferred in fixative solution of 4% Paraformaldehyde Fixative (PAF; Ref. 1.04003.5000, Merck, France) solution for further paraffin-embedding and sebaceous gland size analysis.

Three skin biopsies were collected from rats treated with vehicle in order to evaluate the rate of lipid extraction from epidermis.

Incubation with Radioelements

The biopsies (from 175 to 466 mg) were incubated in 1 ml of KRB in presence of 5 μCi D-[U-¹⁴C]-Dextrose/ml at 37° C., 5% CO₂ for 3 h.

Three additional skin biopsies were collected from rats treated with vehicle and were incubated in KRB at 37° C., 5% CO₂ for 3 h. At the end of incubation, 5 μCi [4-¹⁴C]-Cholesterol/ml were added. This sample allowed calculating the non specific radioactivity.

### Separation of Dermis and Epidermis

After incubation, biopsies were rinsed 3 times with 3 ml of KRB. Dermis and epidermis were separated according to method described by Levang A. K. et al. [4].

So, skin biopsies were soaked in water at 60° C. for 45 s. The skin was then removed and blotted dry and pinned with the dermal skin side down. The epidermis and dermis were separated by scrapping and epidermis was weighted.

Extraction of Lipids from Epidermis

Each fraction (100 mg) epidermis was cut into small fragments by using scalpels. Fragments were homogenized with chloroform:methanol [(2:1; v:v), Ref. C2432, batch No. 055K0070, Sigma, France: Ref M3641-1L, batch No. 104K3641, Sigma, France] according to the ratio 50 mg of tissue/ml of chloroform:methanol (2:1; v:v) with Potter-Elvehjem type of homogenizer [5] (Ref. 48780, Dutscher, France).

For extraction controls, 5 μCi [4-¹⁴C]-Cholesterol/ml was added to the epidermis just before the end of incubation.

Then, the mixture was washed with 0.2 volume of water. Two phases were separated by centrifugation during 20 min at 800 g. The volume of each phase was 40% for the superior phase (aqueous) and 60% for the inferior one (organic). The superior phase was eliminated very carefully and without disturbing inferior phase.

The inferior phase was transferred in scintillating tube (Ref. 720-0494, Amersham, France) for counting radioactivity in 15 ml of scintillating liquid (Ref. NOCS104, batch No. A4340, Amersham, France) with a liquid scintillation counter (Beckman, France).

The specific activity of the radiolabeled dextrose was evaluated by adding 1 μl of D-U-¹⁴C-Dextrose in 1 ml of chloroform:methanol (2:1; v:v) and 15 ml of scintillating liquid. The radioactivity counting (cpm) allowed determining the specific activity as cpm/nmole.

The specific activity of the radiolabeled cholesterol was evaluated by adding 5 μl of [4-¹⁴C]Cholesterol in 1 ml of chloroform:methanol (2:1; v:v) and 15 ml of scintillating liquid. The radioactivity counting (cpm) allowed determining the specific activity as cpm/nmole.

### Study of Sebaceous Gland Size

The size of sebaceous gland was evaluated by histological study of biopsies.

Biopsies fixed in PAF 4% were paraffin-embedded by the ASP 300 (Leica) automat according to the following procedure:

| | | | Temperature |
|---|---|---|---|
| Step | Reagents | Time (h) | (° C.) |
| 1 | Formol (Ref. 1.04003.5000, | 02:00 | 35 |
| | Merck) | | |
| 2 | Alcohol 100% (Ref. 02855, | 00:15 | 35 |
| | Sigma) | | |
| 3 | Alcohol 100% | 00:30 | 35 |
| 4 | Alcohol 100% | 00:45 | 35 |
| 5 | Alcohol 100% | 00:45 | 35 |
| 6 | Alcohol 100% | 01:15 | 35 |
| 7 | Alcohol 100% | 01:45 | 35 |
| 8 | Toluene (Ref. UN 1294, VWR) | 00:30 | 35 |
| 9 | Toluene | 00:45 | 35 |
| 10 | Toluene | 01:00 | 35 |
| 11 | Parrafin (Ref. 6774060, | 00:45 | 60 |
| | Shandon Histoplasta) | | |
| 12 | Parafin | 01:15 | 60 |
| 13 | Parafin | 01:45 | 60 |

Histological sections of biopsies were realized (5 sections/biopsy).

Each one was colored by Hemalun/Eosin/Safran OTTIX coloration method according to the following protocol:

| Step | Reagents | Time (min) |
|---|---|---|
| 1 | OTTIX (Ref. T/02070059, MMFrance) | 05:30 |
| 2 | OTTIX | 05:30 |
| 3 | OTTIX | 05:30 |
| 4 | Absolute alcohol | 03:12 |
| 5 | Wash | 02:12 |
| 6 | Hemalun | 02:12 |
| 7 | Wash | 02:12 |
| 8 | Shandon Bluing Reagent (Ref. 6769001, Thermo | 01:06 |
| | Electron) | |
| 9 | Wash | 02:12 |
| 10 | Eosin y aqueous (Ref. 6766009, Thermo Electron) | 02:12 |
| 11 | Wash | 01:06 |
| 12 | Absolute alcohol | 02:18 |
| 13 | Safran | 01:06 |
| 14 | Absolute alcohol | 01:06 |
| 15 | OTTIX | 01:06 |
| 16 | OTTIX | 03:00 |

A microscopic observation by using a CKX41 light microscope (Olympus, France) was performed. Each sebaceous gland within 1 section/biopsy was numerized with a digital camera (Pwershot A80, Canon) and was measured by using ImageJ software 1.32j.

### Analysis of Lipogenesis

The specific activity of the radiolabeled dextrose was evaluated by adding 1 μl of D-[U-¹⁴C]-Dextrose in 1 ml of chloroform:methanol (2:1; v:v) and 15 ml of scintillating liquid. The radioactivity counting (dpm) allowed determining the specific activity as cpm/nmole.

The specific activity of the radiolabeled cholesterol was evaluated by adding 1 μl of [4-¹⁴C]Cholesterol in 1 ml of chloroform:methanol (2:1; v:v) and 15 ml of scintillating liquid.

The rate of lipid extraction was calculated as following:
R=(dpm_{[4-14C]Cholesterol} after extraction)/dpm_{[4-14C]Cholesterol} before extraction×100

It allowed evaluating the rate of lipid extracted in percentage.

The lipogenesis was evaluated as the number of dpm/100 mg tissue.
Lipogenesis=cpm×R×AS (D-[U-¹⁴C]-Dextrose)

### Analysis of Sebaceous Gland Size

Each sebaceous gland within 1 section/biopsy was numerized with a digital camera (Pwershot A80, Canon) and was measured by using ImageJ software 1.32j. The mean of gland size was calculated for each group.

### Statistical Analysis

An Student-t test was performed according to VisualStat® Professional software (Visualstat Computing, USA). This study allowed the comparison between both test substances, the positive control and the vehicle control group.

### Results:

### Effect of Treatment on Rat Body Weight and General Observations

The eighteen Fuzzy rats were treated according to the schedule [(Q1DX5)×4 weeks] with the vehicle (group No. 1; n=3; topical administration), the positive control 13-cis-RA (group No. 2; n=5; 10 mg/kg; per os), the test substances 4-oxo-tretinoin (group No. 3; n=5; doses 0.1, 0.2%; topical administration) and 4-oxo-isotretinoin (group No. 4; n=5; doses 0.1, 0.5%; topical administration). Topical applications (25 μl) were performed on 2-cm² patches on rat skin.

Rats were weighted and randomized at Do as indicated in Table I and during the course of the experiment, they were weighed twice a week

TABLE I
Rat body weight the day of randomization (D0) and at the end of the
treatment period (D25). The Mean Body Weight Change from D0 to
D25 was calculated. Mean Body Weights (g) are expressed as
mean ± SD for each group.

| | Mean Body | Mean Body | Mean Body Weight |
|---|---|---|---|
| | Weight (g) | Weight (g) | Change (g) |
| | At D0 | At D25 | D0-D25 |
| Group No. 1 | 212.3 ± 12.1 | 244.3 ± 10.1 | 32.0 ± 2.0 |
| Group No. 2 | 214.6 ± 10.5 | 243.0 ± 15.2 | 28.4 ± 7.6 |
| Group No. 3 | 212.2 ± 8.0 | 236.0 ± 6.3 | 24.6 ± 8.8 |
| Group No. 4 | 213.8 ± 11.5 | 234.0 ± 10.3 | 20.2 ± 5.8 |

The mean body weight of rats was progressively increased during treatments for every group (from 212.2-214.6 g at Do to 234.0-244.3 g at D25 for the group No. 1), this suggesting that the vehicle was not toxic. However, the mean body weight change in group No. 4 was statistically different from that observed in group No. 1 (20.2±5.8 g and 32.0±2.0 g, respectively, p<0.05). This significant difference appeared between D21 and D25. It was due to an increased mean body weight in group No. 1.

### Study of Lipogenesis

At the end of treatment (D25), rats were anaesthetized, skin biopsies were collected and lipogenesis was assessed. Briefly, biopsies were incubated with ¹⁴C-Dextrose in Krebs-Ringer buffer for 3 h at 37° C., 5% CO₂. After separation of dermis and epidermis, total lipids were extracted from epidermis. Radioactivity was counted using a β-counter.

The results are presented in Table II as mean DPM and mean pmole of ¹⁴C-Dextrose integrated in 100 mg of tissue for each group and dose of test substance.

TABLE II
Study of lipogenesis in skin biopsies after separation of epidermis. Results
are presented as mean DPM and pmole of ¹⁴C-Dextrose integrated
in 100 mg of tissue for each group. ( ): percent of control group (%).

| | | | | Epidermis (100 mg) | |
|---|---|---|---|---|---|
| | | | | | Mean pmole |
| Group Id. | | | | Mean | ¹⁴C- |
| (Animals per | | Test substances | Dose of | DPM ± | dextrose ± |
| group) | | (concentration) | Treatment | SD | SD |
| 1 (n = 3) | | Vehicle | 0.000 mg/ | 7758 ± 1529 | 9.82 ± 1.94 |
| | | | 2 cm² | | (100%) |
| 2 (n = 5) | | 13-cis-RA | 10 mg/kg | 5188 ± 1875 | 6.55 ± 2.38 |
| | | | | | (67%) |
| 3 | A | 4-Oxo-tretinoin | 0.025 mg/ | 7024 ± 1970 | 8.89 ± 2.51 |
| (n = 5) | | (0.1%) | 2 cm² | | (91%) |
| | B | 4-Oxo-tretinoin | 0.050 mg/ | 4993 ± 2219 | 6.30 ± 2.80 |
| | | (0.2%) | 2 cm² | | (64%) |
| 4 | A | 4-Oxo- | 0.025 mg/ | 5499 ± 2599 | 6.95 ± 3.31 |
| (n = 5) | | isotretinoin | 2 cm² | | (71%) |
| | | (0.1%) | | | |
| | B | 4-Oxo- | 0.125 mg/ | 6524 ± 1792 | 8.25 ± 2.28 |
| | | isotretinoin | 2 cm² | | (84%) |
| | | (0.5%) | | | |

In epidermis, the lipogenesis value obtained for the vehicle group (group No. 1) was 9.82±1.94 pmole of ¹⁴C-Dextrose/100 mg. It was significantly decreased (−33%) in the group treated with 13-cis-RA (6.55±2.38 pmole of ¹⁴C-Dextrose/100 mg), as determined by the Student-t test (p<0.05).

In the group treated with the test substance 4-oxo-tretinoin, lipogenesis was decreased at the dose 0.2% (64% of vehicle group; 6.30±2.80 pmole of ¹⁴C-Dextrose/100 mg). A weak decrease was also observed at the dose 0.1% (91% of vehicle group; 9.10±5.23 pmole of ¹⁴C-Dextrose/100 mg).

In the group treated with the test substance 4-oxo-isotretinoin, lipogenesis was decreased at the doses 0.1% (71% of vehicle group; 6.95±3.31 pmole of ¹⁴C-Dextrose/100 mg) and 0.5% (84% of vehicle group; 8.25±2.28 pmole of ¹⁴C-Dextrose/100 mg).

### Study of Sebaceous Gland Size

At the end of treatment (D25; Oct. 13, 2006), rats were anaesthetized, skin biopsies were collected and embedded in paraffin before being stained by an hematoxylin/eosin solution. Biopsies were then observed and numerized by using a microscope. All sebaceous glands within a biopsy were measured.

The results are presented in Table III as mean sebaceous gland size (pixel²) for each group and dose of test substances.

TABLE III
Study of sebaceous gland size in skin biopsies. Results are presented as
mean size in pixel² for each group. ( ): percent of control group (%).

| Group Id. | | | | |
|---|---|---|---|---|
| (Animals per | Test substances | Dose of | | |
| group) | (concentration) | Treatment | | Mean ± SD |
| 1 (n = 3) | Vehicle | 0.000 | mg/2 cm² | 30417 ± 17494 |
| | | | | (100%) |
| 2 (n = 5) | 13-cis-RA | 10 | mg/kg | 25493 ± 17065 |
| | (10 mg/Kg) | | | (83.8%) |
| 4B (n = 5) | 4-Oxo-isotretinoin | 0.025 | mg/2 cm² | 24998 ± 15931 |
| | (0.1%) | | | (82%) |

The treatment with 13-cis-RA allowed decreasing sebaceous gland size when compared to vehicle group (from 30417±17494 to 25493±17065 pixel²). This decreased value was statistically significant according to Student-t test (p<0.05).

For comparison, the mean sebaceous gland size was measured in the group 4B treated with 0.1% 4-oxo-isotretinoin (the lowest dose of treatment). The mean sebaceous gland size was found to be 24988±15931 pixel², a statistically significant decrease when compared to the vehicle group (group No. 1; 30417±17494 pixel²).

### CONCLUSION

Rat body weight increased with any treatment after full completion of the 4 weeks treatment, this suggests that the test substances were not toxic at the concentrations tested.

According to the literature (Puhvel et al., Arc. Dermatol. Res., 277: 395-399, 1885) the effectiveness of topical 13-cis retinoic acid (13-cis RA) as a sebosuppressive agent was evaluated in hairless (“fuzzy”) rats and hairless mice. At nontoxic dosages (i.e., concentrations which induced no body weight loss), topical 13-cis retinoic acid had no detectable sebosuppressive effects in either of these species.

In this study it was shown that 13-cis-retinoic acid oral treatment in hairless (“fuzzy”) rats allowed decreasing lipogenesis in sldn biopsies (epidermis) and reduction of sebaceous gland size. That correlates very well with findings made in humans successfully treated for severe acne with oral 13-cis-RA (e.g. the commercial 13-cis-RA medicament Roaccutane®).

4-oxo-tretinoin and 4-oxo-isotretinoin substantially inhibited lipogenesis in epidermis. Furthermore 4-oxo-isotretinoin as example was shown to allow decreasing the sebaceous gland size at the dose of 0.1%.

These unexpected activities of 4-oxo-tretinoin and 4-oxo-isotretinoin after topical application on both, the lipogenesis inhibition and the reduction of sebaceous glands size, demonstrate the usefulness of 4-oxo-tretinoin and/or 4-oxo-isotretinoin for the topical treatment of severe acne and seborrhoea.

Evaluation of the Irritating Potential of 4-Oxo-Tretinoin and 4-Oxo-Isotretinoin

The following data are indicative of the for retinoids surprisingly low irritating potential of 4-oxo-tretinoin and 4-oxo-isotretinoin:

The cumulative skin irritating potential of 4-oxo-tretinoin and 4-oxo-isotretinoin was investigated in male Wistar rats (n=5). The test compounds were dissolved in acetone/ethanol (1:1) and were administered once daily for 4 weeks (5 days per week) to pre-defined skin areas (approx. 2 cm²) on the back at concentrations of 0, 0.1, 0.5, 1 or 2% (highest concentration for 4-oxo-tretinoin 1.5% due to limited solubility). Observations and examinations included clinical monitoring of skin reaction and histopathological examination of excised skin specimen at study end.

No signs of skin irritation were seen with 4-oxo-isotretinoin at any concentration. With 4-oxo-tretinoin dose-dependent slight to moderate skin irritation was seen at concentrations ≧0.5%, manifesting clinically as reddening (erythema) and occasional scaling. Histopathological examination of treated skin areas revealed concentration-dependent hyperkeratosis, acanthosis and hypertrophy/hyperplasia of sebaceous glands, and occasional necrosis was seen at concentrations ≧1%. In human isotretnoin and tretinoin applied topically as cream showed local irritation at strength of 0.1% and 0.05%.

### Inter-Conversion of the Two 4-Oxo Isomers in the Skin

After topical administration to rats (n=5 per compound) of 1.5% or 2% of 4-oxo-tretinoin and 4-oxo-isotretinoin, respectively, skin and strip levels of 4-oxo-isotretinoin and 4-oxo-tretinoin were determined in serial samples. Irrespective of the compound applied, skin exposure to both 4-oxo-isotretinoin and 4-oxo-tretinoin could be demonstrated showing that an inter-conversion between the two isomers occurres. This inter-conversion of the 4-oxo-isomers in vivo was demonstrated to occur within the skin, not on the skin and not in the formulation.

What is claimed is:

1. A method for the treatment of dermatological disorders wherein such treatment includes at least one of inhibiting lipogenesis in the skin and reducing the size of sebaceous glands, said treatment comprising the topical administration of a compound selected from 4-oxo-tretinoin, 4-oxo-isotretinoin and mixtures thereof to a patient in need thereof.

2. A method in accordance with claim 1, wherein said dermatological condition is at least one of severe acne and seborrhoea.

3. A method in accordance with claim 2, wherein said dermatological condition is severe acne.

4. A method in accordance with claim 3, wherein the severe acne is acne conglobata.

5. A method in accordance with claim 3, wherein the severe acne is nodulocystic acne.

6. A method in accordance with claim 2, wherein the acne is facial acne of a grade of 7 or more according to the Leeds revised acne grading system or a acne on the back or chest of a grade of 5 or more according to said system.

7. A method in accordance with claim 6, wherein the acne is facial acne of a grade of 9 or more according to the Leeds revised acne grading system or acne on the back or chest of a grade of 6 or more according to said system.

8. A method in accordance with claim 1, wherein said compound is 4-oxo-tretinoin.

9. A method in accordance with claim 1, wherein said compound is a mixture of 4-oxo-isotretinoin and 4-oxo-tretinoin.

10. A method in accordance with claim 1, wherein said compound is 4-oxo-isotretinoin.
